(19) United States
(12) Patent Application Publication
(10) Pub. No.: US 2025/0182280 A1
(43) Pub. Date: Jun. 5, 2025

(54) DIAGNOSTIC TOOL FOR REVIEW OF DIGITAL PATHOLOGY IMAGES

(71) Applicants: Genentech, Inc., South San Francisco, CA (US); Hoffmann-La Roche Inc., Little Falls, NJ (US)

(72) Inventors: Fabien GAIRE, Starnberg (DE); Konstanty KORSKI, Basel (CH); Trung Kien NGUYEN, Chelsea, MI (US); Jacob GILDENBLAT, Holon (IL)

(21) Appl. No.: 19/043,123

(22) Filed: Jan. 31, 2025

Related U.S. Application Data
(63) Continuation of application No. PCT/US2023/071548, filed on Aug. 2, 2023.
(60) Provisional application No. 63/394,928, filed on Aug. 3, 2022.

(51) Int. Cl. G06T 7/00; G06T 7/11; G16H 50/20

(52) U.S. Cl. CPC G06T 7/0012; G06T 7/11; G16H 50/20; G06T 2207/30024; G06T 2207/30096

(57) ABSTRACT

In one embodiment, a method includes accessing a slide image associated with a tissue for a medical analysis, segmenting the slide image into a plurality of tiles, selecting one or more tiles from the plurality of tiles by one or more machine-learning models based on one or more criteria associated with the medical analysis, displaying the one or more selected tiles for user review via a user interface, receiving one or more user inputs associated with the one or more tiles via the user interface, and generating an analysis result for the medical analysis based on the one or more user inputs and the one or more tiles.

### CROSS-REFERENCE TO RELATED APPLICATION

This application is continuation of International Application No. PCT/US2023/071548, filed on Aug. 2, 2023, which claims priority to U.S. Provisional Application No. 63/394,928, filed Aug. 3, 2022, the disclosure of which is hereby incorporated by reference in its entirety.

### FIELD OF THE INVENTION

The present disclosure relates to a system and methods for improving efficiency of medical analysis based on slide images of tissues.

### INTRODUCTION

Pathologists may help with medical analysis of patients by analyzing slide images taken from patients' tissues. These slides images are usually stained, and pathologists are familiar in the review of specific color schemes for immunohistochemistry (IHC) and histological stains. The pathologists may survey these images, select specific areas of each slide image, perform detailed zooming-in, and then analyze these areas. In cancer treatment, pathologists may analyze areas based on specific representative areas of the tumor tissues during the medical analysis of patients with cancer. For example, diffuse large B-cell lymphoma, or DLBCL, is a cancer that starts in white blood cells called lymphocytes. It usually grows in lymph nodes, i.e., the pea-sized glands in the neck, groin, armpits, and elsewhere that are part of a human's immune system. It can also show up in other areas of the body. DLBCL presents relatively homogeneous patterns reflecting clonal growth of the disease. In DLBCL, a large part of the diagnosis is relying on analysis of nuclei (e.g., size, density, content). The analysis results from the pathologists may be provided to doctors to help determine the treatments. To perform the analysis, pathologists usually have to analyze thousands of image tiles, which is very time-consuming and inefficient.

### Summary of Particular Embodiments

Herein is provided a system and methods for improving efficiency of medical analysis based on slide images of tissues.

In particular embodiments, a digital pathology image processing system may improve the efficiency of the medical diagnosis workflow of patients based on slide images taken from their tissues. The digital pathology image processing system may show a pathologist, via a user interface of a software tool associated with the system, an entire tissue image together with a gallery of filtered tiles generated from the tissue image. These filtered tiles may be determined by an algorithm, where only high-attention tiles comprising positive area for a target diagnosis (e.g., tumor) may be shown. On those tiles, the digital pathology image processing system may further generate segmentations (e.g., nuclei for tumor diagnosis). The digital pathology image processing system may then provide, via the software tool, an option to the pathologist to invalidate inappropriate tiles, e.g., with artifacts or with improper segmentation. If a tile is invalidated by the pathologist, another tile may be proposed by the digital pathology image processing system. Once the pathologist has reviewed and marked (e.g., as approved or invalid) a set of tiles via the software tool, the digital pathology image processing system may further generate an analysis result (e.g., inferring a risk score associated with a patient's health to predict recurrence of the illness or resistance to treatment). The analysis result may be further presented to the pathologist for review via the software tool. Once pathologist approval of the analysis result is obtained, the digital pathology image processing system may further generate a report comprising the analysis result. The report may include a prompt or request for the pathologist to sign off, after which the report may be sent to other parties of interest (e.g., clinics, hospitals, doctors, etc.) to assist in a determination of suitable treatment plans for the patient.

In particular embodiments, a digital pathology image processing system may access a slide image associated with a tissue for a medical analysis. The digital pathology image processing system may then segment the slide image into a plurality of tiles. The digital pathology image processing system may then select, by one or more machine-learning models based on one or more criteria associated with the medical analysis, one or more tiles from the plurality of tiles. In particular embodiments, the digital pathology image processing system may display, via a user interface, the one or more selected tiles for user review. The digital pathology image processing system may then receive, via the user interface, one or more user inputs associated with the one or more tiles. The digital pathology image processing system may further generate, based on the one or more user inputs and the one or more tiles, an analysis result for the medical analysis.

### BRIEF DESCRIPTION OF THE DRAWINGS

FIG. 1 illustrates a network of interacting computer systems that can be used, as described herein according to some embodiments of the present disclosure.

FIG. 2 illustrates an example method for facilitating tile review for medical analysis.

FIG. 3 illustrates an example workflow for determining treatment for a patient.

FIG. 4 illustrates an example initial interface of the software tool for reviewing tiles.

FIG. 5 illustrates an example zooming in of a tile.

FIG. 6 illustrates an example tile under review by the pathologist.

FIG. 7 illustrates another example tile under review by the pathologist.

FIG. 8 illustrates an example approval of a tile by the pathologist.

FIG. 9 illustrates an example rejection of a tile by the pathologist.

FIG. 10 illustrates an example of minimum number of approved tiles being reached.

FIG. 11 illustrates an example report generated by the digital pathology image processing system.

FIG. 12 illustrates an example of a computing system.

### DESCRIPTION

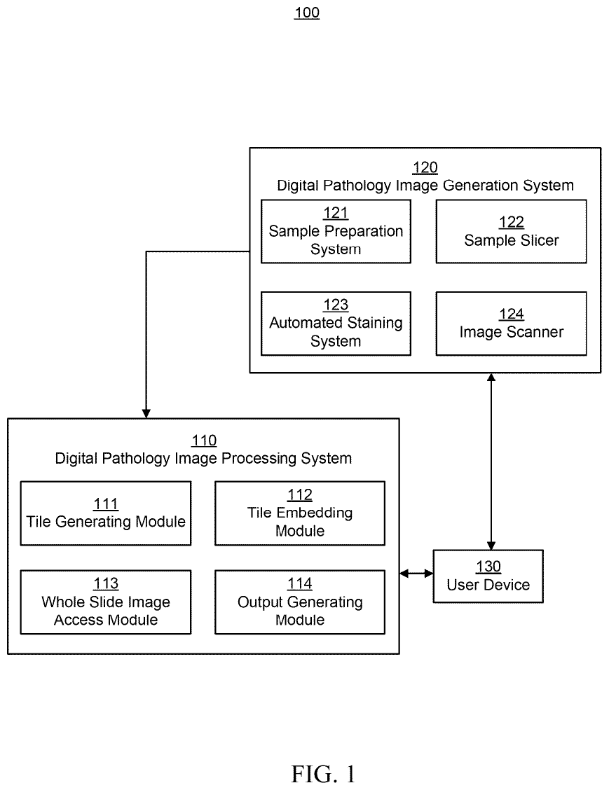

FIG. 1 illustrates a network 100 of interacting computer systems that can be used, as described herein according to some embodiments of the present disclosure.

A digital pathology image generation system 120 can generate one or more whole slide images or other related digital pathology images, corresponding to a particular sample. For example, an image generated by digital pathology image generation system 120 can include a stained section of a biopsy sample. As another example, an image generated by digital pathology image generation system 120 can include a slide image (e.g., a blood film) of a liquid sample. As another example, an image generated by digital pathology image generation system 120 can include fluorescence microscopy such as a slide image depicting fluorescence in situ hybridization (FISH) after a fluorescent probe has been bound to a target DNA or RNA sequence.

Some types of samples (e.g., biopsies, solid samples and/or samples including tissue) can be processed by a sample preparation system 121 to fix and/or embed the sample. Sample preparation system 121 can facilitate infiltrating the sample with a fixating agent (e.g., liquid fixing agent, such as a formaldehyde solution) and/or embedding substance (e.g., a histological wax). For example, a sample fixation sub-system can fix a sample by exposing the sample to a fixating agent for at least a threshold amount of time (e.g., at least 3 hours, at least 6 hours, or at least 13 hours). A dehydration sub-system can dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system can infiltrate the sample (e.g., one or more times for corresponding predefined time periods) with a heated (e.g., and thus liquid) histological wax. The histological wax can include a paraffin wax and potentially one or more resins (e.g., styrene or polyethylene). The sample and wax can then be cooled, and the wax-infiltrated sample can then be blocked out.

A sample slicer 122 can receive the fixed and embedded sample and can produce a set of sections. Sample slicer 122 can expose the fixed and embedded sample to cool or cold temperatures. Sample slicer 122 can then cut the chilled sample (or a trimmed version thereof) to produce a set of sections. Each section can have a thickness that is (for example) less than 100 μm, less than 50 μm, less than 10 μm or less than 5 μm. Each section can have a thickness that is (for example) greater than 0.1 μm, greater than 1 μm, greater than 2 μm or greater than 4 μm. The cutting of the chilled sample can be performed in a warm water bath (e.g., at a temperature of at least 30° C., at least 35° C. or at least 40° C.).

An automated staining system 123 can facilitate staining one or more of the sample sections by exposing each section to one or more staining agents. Each section can be exposed to a predefined volume of staining agent for a predefined period of time. In some instances, a single section is concurrently or sequentially exposed to multiple staining agents.

Each of one or more stained sections can be presented to an image scanner 124, which can capture a digital image of the section. Image scanner 124 can include a microscope camera. The image scanner 124 can capture the digital image at multiple levels of magnification (e.g., using a 10× objective, 20× objective, 40× objective, etc.). Manipulation of the image can be used to capture a selected portion of the sample at the desired range of magnifications. Image scanner 124 can further capture annotations and/or morphometrics identified by a human operator. In some instances, a section is returned to automated staining system 123 after one or more images are captured, such that the section can be washed, exposed to one or more other stains, and imaged again. When multiple stains are used, the stains can be selected to have different color profiles, such that a first region of an image corresponding to a first section portion that absorbed a large amount of a first stain can be distinguished from a second region of the image (or a different image) corresponding to a second section portion that absorbed a large amount of a second stain.

It will be appreciated that one or more components of digital pathology image generation system 120 can, in some instances, operate in connection with human operators. For example, human operators can move the sample across various sub-systems (e.g., of sample preparation system 121 or of digital pathology image generation system 120) and/or initiate or terminate operation of one or more sub-systems, systems, or components of digital pathology image generation system 120. As another example, part or all of one or more components of digital pathology image generation system (e.g., one or more subsystems of the sample preparation system 121) can be partly or entirely replaced with actions of a human operator.

Further, it will be appreciated that, while various described and depicted functions and components of digital pathology image generation system 120 pertain to processing of a solid and/or biopsy sample, other embodiments can relate to a liquid sample (e.g., a blood sample). For example, digital pathology image generation system 120 can receive a liquid-sample (e.g., blood or urine) slide that includes a base slide, smeared liquid sample and cover. Image scanner 124 can then capture an image of the sample slide. Further embodiments of the digital pathology image generation system 120 can relate to capturing images of samples using advancing imaging techniques, such as FISH, described herein. For example, once a florescent probe has been introduced to a sample and allowed to bind to a target sequence appropriate imaging can be used to capture images of the sample for further analysis.

A given sample can be associated with one or more users (e.g., one or more physicians, laboratory technicians and/or medical providers) during processing and imaging. An associated user can include, by way of example and not of limitation, a person who ordered a test or biopsy that produced a sample being imaged, a person with permission to receive results of a test or biopsy, or a person who conducted analysis of the test or biopsy sample, among others. For example, a user can correspond to a physician, a pathologist, a clinician, or a subject. A user can use one or one user devices 130 to submit one or more requests (e.g., that identify a subject) that a sample be processed by digital pathology image generation system 120 and that a resulting image be processed by a digital pathology image processing system 110.

Digital pathology image generation system 120 can transmit an image produced by image scanner 124 back to user device 130. User device 130 then communicates with the digital pathology image processing system 110 to initiate automated processing of the image. In some instances, digital pathology image generation system 120 provides an image produced by image scanner 124 to the digital pathology image processing system 110 directly, e.g., at the direction of the user of a user device 130. Although not illustrated, other intermediary devices (e.g., data stores of a server connected to the digital pathology image generation system 120 or digital pathology image processing system 110) can also be used. Additionally, for the sake of simplicity only one digital pathology image processing system 110, image generating system 120, and user device 130 is illustrated in the network 100. This disclosure anticipates the use of one or more of each type of system and component thereof without necessarily deviating from the teachings of this disclosure.

The network 100 and associated systems shown in FIG. 1 can be used in a variety of contexts where scanning and evaluation of digital pathology images, such as whole slide images, are an essential component of the work. As an example, the network 100 can be associated with a clinical environment, where a user is evaluating the sample for possible diagnostic purposes. The user can review the image using the user device 130 prior to providing the image to the digital pathology image processing system 110. The user can provide additional information to the digital pathology image processing system 110 that can be used to guide or direct the analysis of the image by the digital pathology image processing system 110. For example, the user can provide a prospective diagnosis or preliminary assessment of features within the scan. The user can also provide additional context, such as the type of tissue being reviewed. As another example, the network 100 can be associated with a laboratory environment were tissues are being examined, for example, to determine the efficacy or potential side effects of a drug. In this context, it can be commonplace for multiple types of tissues to be submitted for review to determine the effects on the whole body of said drug. This can present a particular challenge to human scan reviewers, who may need to determine the various contexts of the images, which can be highly dependent on the type of tissue being imaged. These contexts can optionally be provided to the digital pathology image processing system 110.

Digital pathology image processing system 110 can process digital pathology images, including whole slide images, to classify the digital pathology images and generate annotations for the digital pathology images and related output. As an example, the digital pathology image processing system 110 can process whole slide images of tissue samples or tiles of the whole slide images of tissue samples generated by the digital pathology image processing system 110, to identify tumor regions. As another example, the digital pathology image processing system 110 can process tiles of the whole slide images of tissue samples to identify tiles with high-attention values or high-risk scores associated with a medical analysis. The digital pathology image processing system 110 may crop the querying image into a plurality of image tiles. A tile generating module 111 can define a set of tiles for each digital pathology image. To define the set of tiles, the tile generating module 111 can segment the digital pathology image into the set of tiles. As embodied herein, the tiles can be non-overlapping (e.g., each tile includes pixels of the image not included in any other tile) or overlapping (e.g., each tile includes some portion of pixels of the image that are included in at least one other tile). Features such as whether or not tiles overlap, in addition to the size of each tile and the stride of the window (e.g., the image distance or pixels between a tile and a subsequent tile) can increase or decrease the data set for analysis, with more tiles (e.g., through overlapping or smaller tiles) increasing the potential resolution of eventual output and visualizations. In some instances, tile generating module 111 defines a set of tiles for an image where each tile is of a predefined size and/or an offset between tiles is predefined. Continuing with the example of detecting gene fusions, each slide image may be cropped into image tiles with width and height of certain number of pixels. Furthermore, the tile generating module 111 can create multiple sets of tiles of varying size, overlap, step size, etc., for each image. As an example, the width and height of pixels may be dynamically determined (i.e., not fixed) based on factors such as the evaluation task, the querying image itself, or any suitable factor. In some embodiments, the digital pathology image itself can contain tile overlap, which may result from the imaging technique. Even segmentation without tile overlapping can be a preferable solution to balance tile processing requirements and avoid influencing the embedding generation and weighting value generation discussed herein. A tile size or tile offset can be determined, for example, by calculating one or more performance metrics (e.g., precision, recall, accuracy, and/or error) for each size/offset and by selecting a tile size and/or offset associated with one or more performance metrics above a predetermined threshold and/or associated with one or more performance metric(s) (e.g., high precision, high recall, high accuracy, and/or low error).

The tile generating module 111 may further define a tile size depending on the type of abnormality being detected. For example, the tile generating module 111 can be configured with awareness of the type(s) of tissue abnormalities that the digital pathology image processing system 110 will be searching for and can customize the tile size according to the tissue abnormalities to improve detection. For example, the image generating module 111 can determine that, when the tissue abnormalities include searching for inflammation or necrosis in lung tissue, the tile size should be reduced to increase the scanning rate, while when the tissue abnormalities include abnormalities with Kupffer cells in liver tissues, the tile size should be increased to increase the opportunities for the digital pathology image processing system 110 to analyze the Kupffer cells holistically. In some instances, tile generating module 111 defines a set of tiles where a number of tiles in the set, size of the tiles of the set, resolution of the tiles for the set, or other related properties, for each image is defined and held constant for each of one or more images.

As embodied herein, the tile generating module 111 can further define the set of tiles for each digital pathology image along one or more color channels or color combinations. As an example, digital pathology images received by digital pathology image processing system 110 can include large-format multi-color channel images having pixel color values for each pixel of the image specified for one of several color channels. Example color specifications or color spaces that can be used include the RGB, CMYK, HSL, HSV, or HSB color specifications. The set of tiles can be defined based on segmenting the color channels and/or generating a brightness map or greyscale equivalent of each tile. For example, for each segment of an image, the tile generating module 111 can provide a red tile, blue tile, green tile, and/or brightness tile, or the equivalent for the color specification used. As explained herein, segmenting the digital pathology images based on segments of the image and/or color values of the segments can improve the accuracy and recognition rates of the networks used to generating embeddings for the tiles and image and to produce classifications of the image. Additionally, the digital pathology image processing system 110, e.g., using tile generating module 111, can convert between color specifications and/or prepare copies of the tiles using multiple color specifications. Color specification conversions can be selected based on a desired type of image augmentation (e.g., accentuating or boosting particular color channels, saturation levels, brightness levels, etc.). Color specification conversions can also be selected to improve compatibility between digital pathology image generation systems 120 and the digital pathology image processing system 110. For example, a particular image scanning component can provide output in the HSL color specification and the models used in the digital pathology image processing system 110, as described herein, can be trained using RGB images. Converting the tiles to the compatible color specification can ensure the tiles can still be analyzed. Additionally, the digital pathology image processing system can up-sample or down-sample images that are provided in particular color depth (e.g., 8-bit, 1-bit, etc.) to be usable by the digital pathology image processing system. Furthermore, the digital pathology image processing system 110 can cause tiles to be converted according to the type of image that has been captured (e.g., fluorescent images may include greater detail on color intensity or a wider range of colors).

As described herein, a tile embedding module 112 can generate an embedding for each tile in a corresponding feature embedding space. The embedding can be represented by the digital pathology image processing system 110 as a feature vector for the tile. The tile embedding module 112 can use a neural network (e.g., a convolutional neural network) to generate a feature vector that represents each tile of the image. In particular embodiments, the tile embedding neural network can be based on the ResNet image network trained on a dataset based on natural (e.g., non-medical) images, such as the ImageNet dataset. By using a non-specialized tile embedding network, the tile embedding module 112 can leverage known advances in efficiently processing images to generating embeddings. Furthermore, using a natural image dataset allows the embedding neural network to learn to discern differences between tile segments on a holistic level.

In other embodiments, the tile embedding network used by the tile embedding module 112 can be an embedding network customized to handle large numbers of tiles of large format images, such as digital pathology whole slide images. Additionally, the tile embedding network used by the tile embedding module 112 can be trained using a custom dataset. For example, the tile embedding network can be trained using a variety of samples of whole slide images or even trained using samples relevant to the subject matter for which the embedding network will be generating embeddings (e.g., scans of particular tissue types). Training the tile embedding network using specialized or customized sets of images can allow the tile embedding network to identify finer differences between tiles which can result in more detailed and accurate distances between tiles in the feature embedding space at the cost of additional time to acquire the images and the computational and economic cost of training multiple tile generating networks for use by the tile embedding module 112. The tile embedding module 112 can select from a library of tile embedding networks based on the type of images being processed by the digital pathology image processing system 110.

As described herein, tile embeddings can be generated from a deep learning neural network using visual features of the tiles. Tile embeddings can be further generated from contextual information associated with the tiles or from the content shown in the tile. For example, a tile embedding can include one or more features that indicate and/or correspond to a size of depicted objects (e.g., sizes of depicted cells or aberrations) and/or a density of depicted objects (e.g., a density of depicted cells or aberrations). Size and density can be measured absolutely (e.g., width expressed in pixels or converted from pixels to nanometers) or relative to other tiles from the same digital pathology image, from a class of digital pathology images (e.g., produced using similar techniques or by a single digital pathology image generation system or scanner), or from a related family of digital pathology images. Furthermore, tiles can be classified prior to the tile embedding module 112 generating embeddings for the tiles such that the tile embedding module 112 considers the classification when preparing the embeddings.

For consistency, the tile embedding module 112 may produce embeddings of a predefined size (e.g., vectors of 512 elements, vectors of 2048 bytes, etc.). The tile embedding module 112 can also produce embeddings of various and arbitrary sizes. The tile embedding module 112 can adjust the sizes of the embeddings based on user direction or can be selected, for example, based on computation efficiency, accuracy, or other parameters. In particular embodiments, the embedding size can be based on the limitations or specifications of the deep learning neural network that generated the embeddings. Larger embedding sizes can be used to increase the amount of information captured in the embedding and improve the quality and accuracy of results, while smaller embedding sizes can be used to improve computational efficiency.

The digital pathology image processing system 110 can perform different inferences by apply one or more machine-learning models to the embeddings, i.e., inputting the embeddings to a machine-learning model. As an example, the digital pathology image processing system 110 can identify, based on a machine-learning model trained to identify tumor regions, a tumor region. As another example, the digital pathology image processing system 110 can identify, based on a machine-learning model trained to identify high-attention or high-risk tiles, a high-attention or high-risk tile. In some embodiments, it may be not necessary to crop the image into image tiles, generate embeddings for these tiles, and then perform inferences based on such embeddings. Instead, the digital pathology image processing system 110 can directly apply the machine-learning model to the embedding of a whole slide image to make inference with sufficient GPU memory. The output of the machine-learning model may be resized into the shape of the input image.

A whole slide image access module 113 can manage requests to access whole slide images from other modules of the digital pathology image processing system 110 and the user device 130. For example, the whole slide image access module 113 receive requests to identify a whole slide image based on a particular tile, an identifier for the tile, or an identifier for the whole slide image. The whole slide image access module 113 can perform tasks of confirming that the whole slide image is available to the user requesting, identifying the appropriate databases from which to retrieve the requested whole slide image, and retrieving any additional metadata that may be of interest to the requesting user or module. Additionally, the whole slide image access module 113 can handle efficiently streaming the appropriate data to the requesting device. As described herein, whole slide images may be provided to user devices in chunks, based on the likelihood that a user will wish to see the portion of the whole slide image. The whole slide image access module 113 can determine which regions of the whole slide image to provide and determine how to provide them. Furthermore, the whole slide image access module 113 can be empowered within the digital pathology image processing system 110 to ensure that no individual component locks up or otherwise misuses a database or whole slide image to the detriment of other components or users.

An output generating module 114 of the digital pathology image processing system 110 can generate an output corresponding to result tile and result whole slide image datasets based on user request. As described herein, the output can include a variety of visualizations, interactive graphics, and reports based upon the type of request and the type of data that is available. In many embodiments, the output will be provided to the user device 130 for display, but in certain embodiments the output can be accessed directly from the digital pathology image processing system 110. The output will be based on an existence of and access to the appropriate data, so the output generating module will be empowered to access necessary metadata and anonymized patient information as needed. As with the other modules of the digital pathology image processing system 110, the output generating module 114 can be updated and improved in a modular fashion, so that new output features can be provided to users without requiring significant downtime.

The general techniques described herein can be integrated into a variety of tools and use cases. For example, as described, a user (e.g., pathology or clinician) can access a user device 130 that is in communication with the digital pathology image processing system 110 and provide a query image for analysis. The digital pathology image processing system 110, or the connection to the digital pathology image processing system can be provided as a standalone software tool or package that searches for corresponding matches, identifies similar features, and generates appropriate output for the user upon request. As a standalone tool or plug-in that can be purchased or licensed on a streamlined basis, the tool can be used to augment the capabilities of a research or clinical lab. Additionally, the tool can be integrated into the services made available to the customer of digital pathology image generation systems. For example, the tool can be provided as a unified workflow, where a user who conducts or requests a whole slide image to be created automatically receives a report of noteworthy features within the image and/or similar whole slide images that have been previously indexed. Therefore, in addition to improving whole slide image analysis, the techniques can be integrated into existing systems to provide additional features not previously considered or possible.

Moreover, the digital pathology image processing system 110 can be trained and customized for use in particular settings. For example, the digital pathology image processing system 110 can be specifically trained for use in providing insights relating to specific types of tissue (e.g., lung, heart, blood, liver, etc.). As another example, the digital pathology image processing system 110 can be trained to assist with safety assessment, for example in determining levels or degrees of toxicity associated with drugs or other potential therapeutic treatments. Once trained for use in a specific subject matter or use case, the digital pathology image processing system 110 is not necessarily limited to that use case. Training may be performed in a particular context, e.g., toxicity assessment, due to a relatively larger set of at least partially labeled or annotated images.

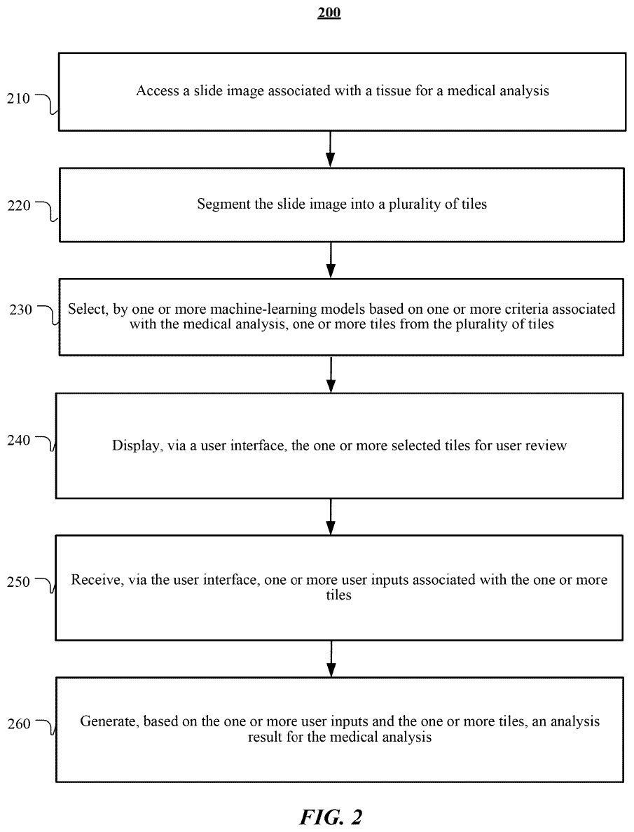

FIG. 2 illustrates an example method 200 for facilitating tile review for medical analysis. The method may begin at step 210, where the digital pathology image processing system 110 may access a slide image associated with a tissue for a medical analysis. In particular embodiments, such slide image may correspond to a stained slide taken from the patient's tissue(s). The reason for staining the slide may be because detection of objects may require a familiar color pattern for pathologists for higher efficiency. As an example and not by way of limitation, the slides may be stained by hematoxylin and eosin (H&E). The stained slides may be then digitalized (e.g., scanned) to generate slide images.

At step 220, the digital pathology image processing system 110 may segment the slide image into a plurality of tiles. In particular embodiments, the tile generating module 111 may be used to generate the tiles. The tiles may be non-overlapping or overlapping. Features such as whether or not tiles overlap, in addition to the size of each tile and the stride of the window can increase or decrease the data set for analysis, with more tiles increasing the potential resolution of eventual output and visualizations. In particular embodiments, each tile may be of a predefined size and/or an offset between tiles may be predefined. Furthermore, the tile generating module 111 may create multiple sets of tiles of varying size, overlap, step size, etc., for each image. The tile generating module 111 may generate tiles for each digital pathology image along one or more color channels or color combinations. The tiles may be generated based on segmenting the color channels and/or generating a brightness map or greyscale equivalent of each tile. Additionally, the digital pathology image processing system 110 can up-sample or down-sample images that are provided in particular color depth to be usable by the digital pathology image processing system 110. Furthermore, the digital pathology image processing system 110 can cause tiles to be converted according to the type of image that has been captured.

At step 230, the digital pathology image processing system 110 may select, by one or more machine-learning models based on one or more criteria associated with the medical analysis, one or more tiles from the plurality of tiles. In particular embodiments, the digital pathology image processing system 110 may select these tiles as follows. The digital pathology image processing system 110 may filter out tiles of artifacts using a quality-check (QC) algorithm. The digital pathology image processing system 110 may then filter out tiles corresponding to normal areas using a condition-detection algorithm (e.g., if the medical analysis comprises a tumor analysis, non-tumor tiles may be considered “not normal” and therefore, filtered). The digital pathology image processing system 110 may then use another algorithm to pre-select tiles of interest based on different criteria. In particular embodiments, the one or more criteria may comprise one or more of a high-attention value or a high representativeness of an illness targeted by the medical analysis.

At step 240, the digital pathology image processing system 110 may display, via a user interface, the one or more selected tiles for user review. In particular embodiments, the user interface may show the pathologist individual tiles or clusters of tiles. The initial interface may provide an overview of a slide, tile review of the slide, and a slide navigator. As an example, the tile review section may comprise the tiles and the pathologist may scroll down to review other tiles. The digital pathology image processing system 110 may display, via the user interface, locations of the selected tiles with respect to the tissue, respectively. In addition, the user interface may provide different options of visualization. As an example and not by way of limitation, user interface may enable the pathologist to review the tile under different stains. The user interface may be operable for adjusting the display of each of the one or more selected tiles based on one or more of a H & E stain or a virtual stain artificially generated by the digital pathology image processing system 110. For example, the tile may appear as if it had been stained not with H&E, but with a DAB stain (i.e., 3,3′-diaminobenzidine oxidized by hydrogen peroxide in a reaction typically catalyzed by horseradish peroxidase (HRP)). The user interface may also meld or overlay the tile. In addition, the user interface may provide channels that can be activated/deactivated by the pathologist.

At step 250, the digital pathology image processing system 110 may receive, via the user interface, one or more user inputs associated with the one or more tiles. In particular embodiments, the one or more user inputs may comprise one or more of an approval of a tile, a rejection of a tile, or a score for a tile. The pathologist may approve or reject each tile based on the verified input (e.g., properly segmented tumor nuclei). For each rejected tile, the digital pathology image processing system 110 may replace it with another pre-selected tile by the algorithm (e.g., with high-attention value or high representativeness) in order to keep the optimal number of tiles. In alternative embodiments, the pathologist may provide a score for each tile instead of simply approving or rejecting it. Specific instructions with strict criteria with respect to when to approve or reject a tile may be provided to pathologists before the reviewing process is started.

At step 260, the digital pathology image processing system 110 may generate, based on the one or more user inputs and the one or more tiles, an analysis result for the medical analysis. Generating the analysis result may be automatically triggered responsive to determining that the number of the approved tiles reaches the predetermined number. In other words, once the minimum number of tiles is reached, the statistical analysis (e.g., a calculation of a risk score) may be triggered. The digital pathology image processing system 110 may perform the statistical analysis based on the pathologist-approved tiles. Alternatively, the digital pathology image processing system 110 may perform the statistical analysis using the scores of the tiles if the pathologist provides scores to tiles instead of approving/rejecting them. In particular embodiments, the analysis result may comprise one or more of a risk score indicating a likelihood for a recurrence of an illness, a risk score indicating a likelihood for a resistance to a treatment, or a probability indicating a risk of relapse or refractory at a particular time point. Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates an example method for facilitating tile review for medical analysis, including the particular steps of the method of FIG. 2, this disclosure contemplates any suitable method for facilitating tile review for medical analysis, including any suitable steps, which may include all, some, or none of the steps of the method of FIG. 2, where appropriate. Furthermore, although this disclosure describes and illustrates particular components, devices, or systems carrying out particular steps of the method of FIG. 2, this disclosure contemplates any suitable combination of any suitable components, devices, or systems carrying out any suitable steps of the method of FIG. 2.

The medical analysis of slide images associated with tissues may rely on a combination of different factors, such as attention (i.e., is a tile of the slide image important?) and risk score (i.e., is the tile high or low risk?). As an example and not by way of limitation, in DLBCL, it may be challenging to detect tumor areas by an algorithm. The selection may need to be supervised by a pathologist. In addition, the selection of relevant areas (e.g., tiles) may need to allow visualization of histological context with zoom and pan functions. Even though the algorithm can be perceived as a black box, a pathologist may need to sign off on the analysis results, which indicates that it may be important to keep a human (e.g., a pathologist) in the loop to increase confidence of the analysis. Considering the aforementioned factors that are essential for effective analysis of slide images, the embodiments disclosed herein have developed a digital pathology image processing system 110 that integrates machine learning and human expertise for selecting tiles and then generating analysis results based on the selected tiles.

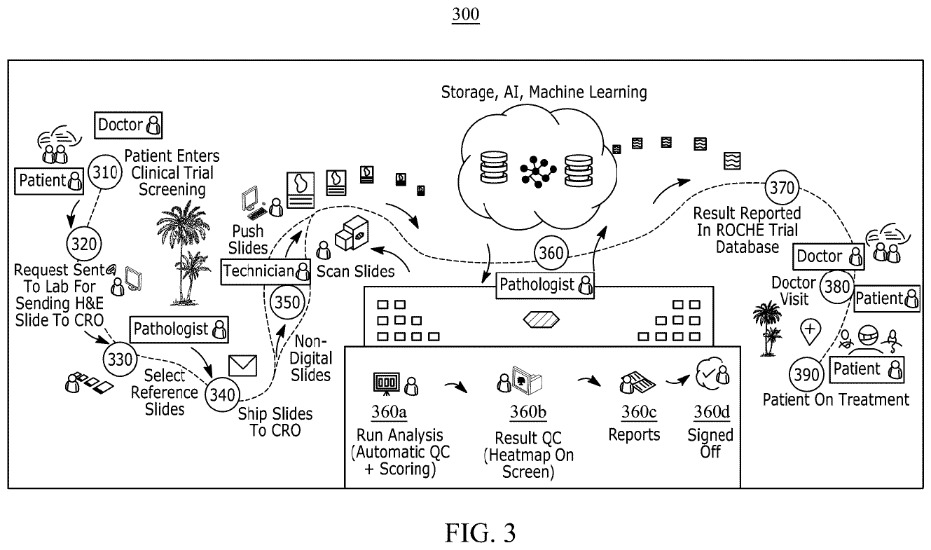

FIG. 3 illustrates an example workflow 300 for determining treatment for a patient. After a patient sees a doctor initially, the patient may enter the clinical trial screening at step 310. In particular embodiments, the medical analysis may comprise determining one or more of a recurrence of an illness or a resistance to a treatment. At step 320, the request for certain testing associated with the patient's illness (e.g., tumor) may be then sent to a lab for sending stained slides taken from the patient's tissue(s) to the clinical research organization (CRO). The reason for staining the slides may be because detection of objects may require a familiar color pattern for pathologists for higher efficiency. As an example and not by way of limitation, the slides may be H & E-stained. At step 330, a pathologist may select reference slides. At step 340, the slides together with the reference slides may be shipped to CRO. At step 350, a technician may push or scan the non-digital slides to digitalize them, i.e., generating slide images.

At step 360, the digital pathology image processing system 110 may store the slide images in storage and then use artificial intelligence (AI) and machine learning to analyze them while keeping a pathologist in the loop based on the following sub-steps. In particular embodiments, the machine-learning models may be based on neural networks. At sub-step 360a, the pathologist may initiate the analysis on one selected slide that is representative (e.g., showing the dominant histological pattern) for the medical case(s) (e.g., tumor) under analysis. The digital pathology image processing system 110 may prepare the slide image accordingly.

At sub-step 360b, the digital pathology image processing system 110 may verify the input. In particular embodiments, the digital pathology image processing system 110 may generate a first subset from the plurality of tiles by filtering out one or more first tiles from the plurality of tiles. Each of the one or more first tiles may comprise an artifact and the selected one or more tiles may be selected from the first subset. Specifically, the digital pathology image processing system 110 may use a quality-check (QC) algorithm to filter out artifacts of the slide images. In particular embodiments, the digital pathology image processing system 110 may generate a second subset from the first subset by filtering out one or more second tiles from the first subset. Each of the one or more second tile may correspond to an area for the medical analysis and the selected one or more tiles may be selected from the second subset. Specifically, the digital pathology image processing system 110 may use a condition-detection algorithm to filter out normal tiles. As an example and not by way of limitation, if the medical analysis comprises a tumor analysis, generating the second subset may be based on a tumor detection algorithm and each of the tiles in the second subset may comprise a tumor.

The digital pathology image processing system 110 may then use another algorithm to pre-select tiles of interest based on different criteria. In particular embodiments, the one or more criteria may comprise one or more of a high-attention value or a high representativeness of an illness targeted by the medical analysis. As an example and not by way of limitation, such algorithm may be based on attention values associated with the tiles, respectively. In particular embodiments, such algorithm may determine tiles with high-attention values are the most impactful and their patterns are the most relevant. For instance, the pre-selected tiles may all have high-attention values. More information on high-attention learning may be found in U.S. Patent Application No. 63/108,659, filed 2 Nov. 2020, which is incorporated by reference in its entirety. As another example and not by way of limitation, the pre-selection of tiles may be based on representativeness of the illness of interest (e.g., tumor). For instance, the pre-selected tiles may all have representativeness. If the tissue is associated with a patient having tumor, the digital pathology image processing system 110 may further generate, for each of the selected tiles, segmentations comprising nuclei. In particular embodiments, the algorithm for pre-selection may be trained based on experts' experience. As an example and not by way of limitation, experts may be asked to annotate a plurality of images indicating what are the most representative areas (e.g., tiles) for the patient and then the algorithm may be trained to find those exact areas. The pathologist may then perform quality check on these tiles that were preselected by the algorithm. In particular embodiments, the digital pathology image processing system 110 may generate a user interface via a software tool to allow the pathologist to easily perform quality check. Besides pathologists, the user interface may be viewable/accessible by any party of interest under clinical disclosure agreement.

In particular embodiments, the user interface may show the pathologist individual tiles or clusters of tiles. The pathologist may review each tile and provide user inputs. In particular embodiments, the one or more user inputs may comprise one or more of an approval of a tile, a rejection of a tile, or a score for a tile. The pathologist may approve or reject each tile based on the verified input (e.g., properly segmented tumor nuclei). In alternative embodiments, the pathologist may provide a score for each tile instead of simply approving or rejecting it. Specific instructions with strict criteria with respect to when to approve or reject a tile may be provide to pathologists before the reviewing process is started. In alternative embodiments, the digital pathology image processing system 110 may further provide annotations of each tile to the pathologist, which may help with the pathologist's review of the tile. The annotations may be generated by an algorithm or by another pathologist.

For each approved tile, the digital pathology image processing system 110 may provide visualizations that can help the pathologist better review it. As an example and not by way of limitation, if the tile is associated with a tumor, the digital pathology image processing system 110 may show nuclei in orange color (e.g., DAB stain), or red stain, or blue stain, and flags based on what a pathologist is used to. If the one or more user inputs comprise one or more rejections of one or more tiles, the digital pathology image processing system 110 may further select, by the one or more machine-learning models based on the one or more criteria associated with the medical analysis, one or more additional tiles from the plurality of tiles for the user review. In other words, for each rejected tile, the digital pathology image processing system 110 may replace it with another tile pre-selected by the algorithm (e.g., with high-attention value or high representativeness) in order to keep the optimal number (e.g., 50) of tiles. In alternative embodiments, the digital pathology image processing system may not replace the rejected tiles. Instead, there may be an excess amount of tiles available for pre-selection. The digital pathology image processing system may continue pre-selecting from this excess amount of the tiles for pathologist to review until the approved tiles by the pathologist are sufficient for generating the analysis results. In particular embodiments, if the one or more user inputs comprise one or more approvals of one or more tiles, the digital pathology image processing system 110 may further determine a number of the one or more approved tiles reaches a predetermined number. Generating the analysis result may be automatically triggered responsive to determining the number of the one or more approved tiles reaches the predetermined number. In other words, once the minimum number of tiles is reached, the statistical analysis (e.g., a calculation of a risk score) may be triggered. In alternative embodiments, generating the analysis result may be automatically triggered responsive to determining a certain area of preselected tiles is reached. As an example and not by way of limitation, there may be a particular area with more importance. The digital pathology image processing may pre-select other areas for the pathologist to review at the beginning. But the process may continue until the digital pathology image processing system pre-select this particular area for the pathologist to review and the pre-selection is approved. The digital pathology image processing system 110 may perform the statistical analysis based on the pathologist-approved tiles. Alternatively, the digital pathology image processing system 110 may perform the statistical analysis using the scores of the tiles if the pathologist provides scores to tiles instead of approving/rejecting them. In particular embodiments, the approval/rejection workflow may minimize clicks by the pathologist, thereby increasing the speed for the pathologist's review.

The digital pathology image processing system 110 may then output the statistical analysis via the software tool to present it to the pathologist for additional review. In particular embodiments, the analysis result may comprise one or more of a risk score indicating a likelihood for a recurrence of an illness, a risk score indicating a likelihood for a resistance to a treatment, or a probability indicating a risk of relapse or refractory at a particular time point.

In particular embodiments, the digital pathology image processing system 110 may receive, via the user interface, one or more additional user inputs comprising one or more of an approval of the analysis result, an adjustment of the analysis result, or an override of the analysis result. Specifically, the pathologist may approve the statistical analysis (e.g., the risk score), which may trigger the generation of the report. Alternatively, the pathologist may adjust the final output, e.g., to adjust the risk score, and even possibly override the final output. The digital pathology image processing system 110 may then generate, based on the one or more additional user inputs, a medical report. At sub-step 360c, the digital pathology image processing system 110 may present the generated report to the pathologist for review. As an example and not by way of limitation, the pathologist may review the report by checking each element (checkboxes) and approve the final report. Subsequently, the digital pathology image processing system 110 may receive a sign-off of the medical report. At sub-step 360d, the digital pathology image processing system 110 may issue an electronic report to the pathologist so that the pathologist can sign it off.

At step 370, the report comprising the analysis result may be stored at a trial database for parties of interest to easily access. At step 380, the patient may visit the doctor again. At step 390, the patient may be put on treatment, which may be determined based on the report comprising the analysis result.

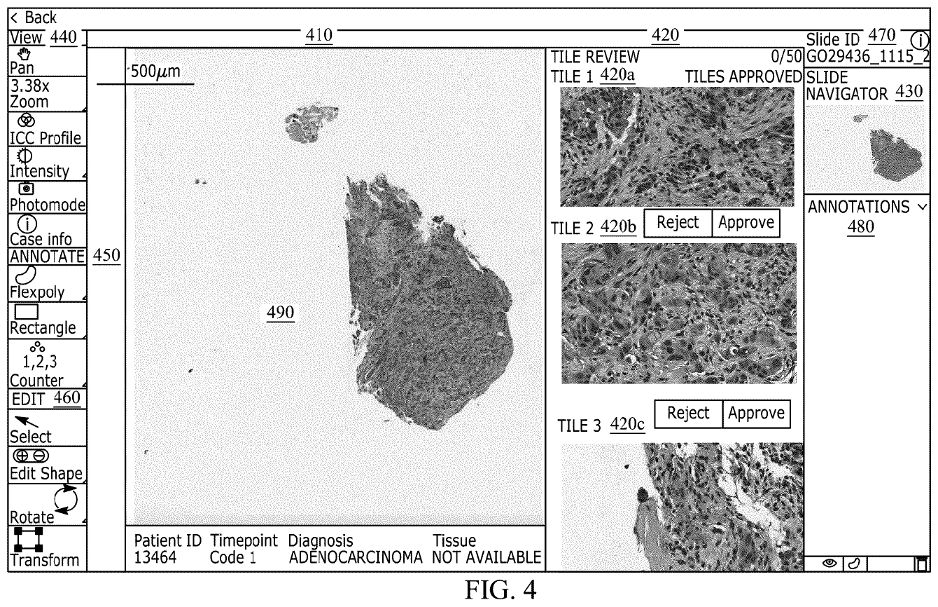

FIG. 4 illustrates an example initial interface of the software tool for reviewing tiles. The initial interface may provide an overview 410 of a slide, tile review section 420 of the slide, and a slide navigator 430. The initial interface may also provide different view options 440. As an example and not by way of limitation, these view options 440 may comprise pan, zoom, ICC profile, intensity, photo mode, and case information (info). The initial interface may additionally provide different annotate options 450. As an example and not by way of limitation, these annotate options 440 may comprise flexpoly, rectangle, and counter. Flexpoly may allow drawing free hand annotations to create any shape that is desired by the user. Rectangle may allow drawing annotations in the form of rectangular of different sizes. Counter may allow annotating any object by putting a mark (e.g., colored dot) in its center, where each dot may be labeled according to the desired need of the user. The initial interface may further provide different edit options 460. As an example and not by way of limitation, these edit options 460 may comprise select, edit shape, rotate, and transform. The user may use these edit options 460 if there is a need to correct or change existing annotation. This correction or change may comprise modification of size, proportions, and location of the existing annotation. In particular embodiments, the initial interface may display the slide identifier (ID) 470 and annotations 480. In particular embodiments, the slide 490 may be stained. As an example and not by way of limitation, the slide 490 in FIG. 4 may be H & E stained. As illustrated in FIG. 4, the tile review section 420 may comprise the tiles and the pathologist may scroll down to review other tiles besides tile 1 420a, tile 2 420b, and tile 3 420c. The pathologist may either reject or approve a tile. The minimum number of tiles to be approved may be 50 and currently there is no tile approved yet (i.e., 0/50).

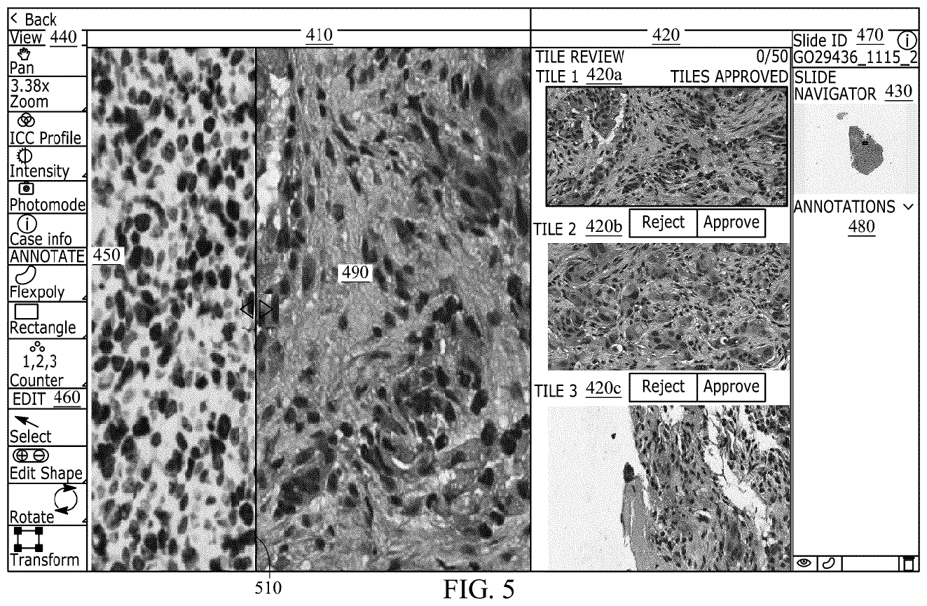

FIG. 5 illustrates an example zooming in of a tile. As illustrated in FIG. 5, the pathologist may be reviewing tile 1 420a. In particular embodiments, the digital pathology image processing system 110 may display, via the user interface, one or more locations of the one or more selected tiles with respect to the tissue, respectively. As illustrated in FIG. 5, the slide navigator 430 may indicate the location of this tile 420a with respect to the whole slide image. In addition, the software tool may provide different options of visualization. In particular embodiments, the digital pathology image processing system 110 may generate a virtual stain (e.g., a virtual DAB stain) associated with the tissue artificially by the one or more machine-learning models based on the information derived from the underlying segmentation results. If the tissue is originally stained based on a H & E stain, the user interface may be operable for adjusting the display of each of the one or more selected tiles based on one or more of the H&E stain or the virtual DAB stain. As an example and not by way of limitation, if all tumor nuclei are detected and segmented by the machine-learning models, the results of the segmentation may be displayed as a virtual DAB stain using the slider function in the user interface. It may not require the registration or overlap of a true DAB stain. As illustrated in FIG. 5, the left part of the tile 420a under review may display a virtual DAB stain whereas the right part of the tile 420a under review may display the original H & E stain. The pathologies may easily slide left and right using the separator 510 to review the same part of the tile 420a under different stains. The software tool may also meld or overlay the tile 420a. In addition, the software tool may provide channels that can be activated/deactivated by the pathologist. As an example and not by way of limitation, with the channels, the software tool may create three different channel locations.

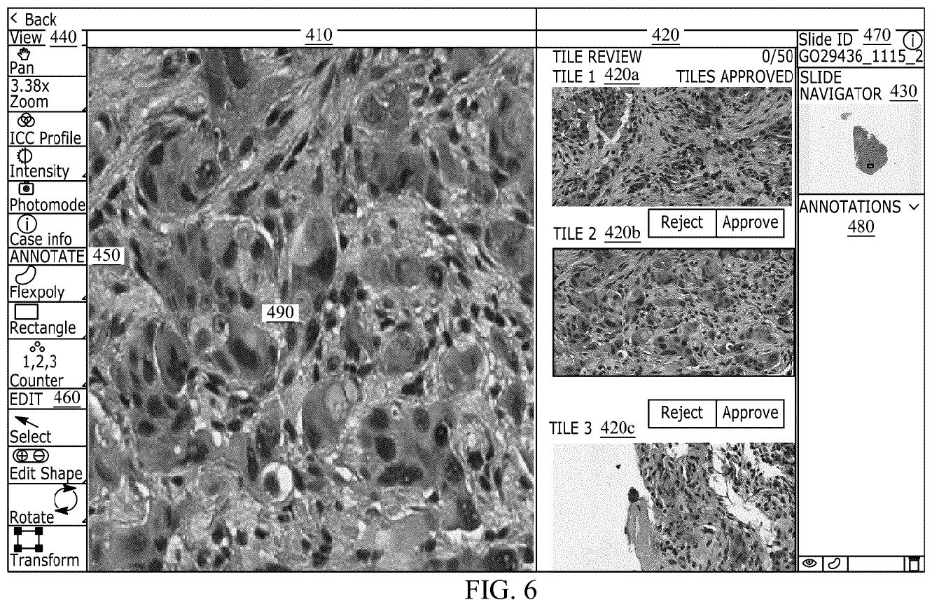

FIG. 6 illustrates an example tile under review by the pathologist. As illustrated in FIG. 6, the pathologist may be reviewing tile 2 420b. The slide navigator 430 may indicate the location of this tile 420b with respect to the whole slide image.

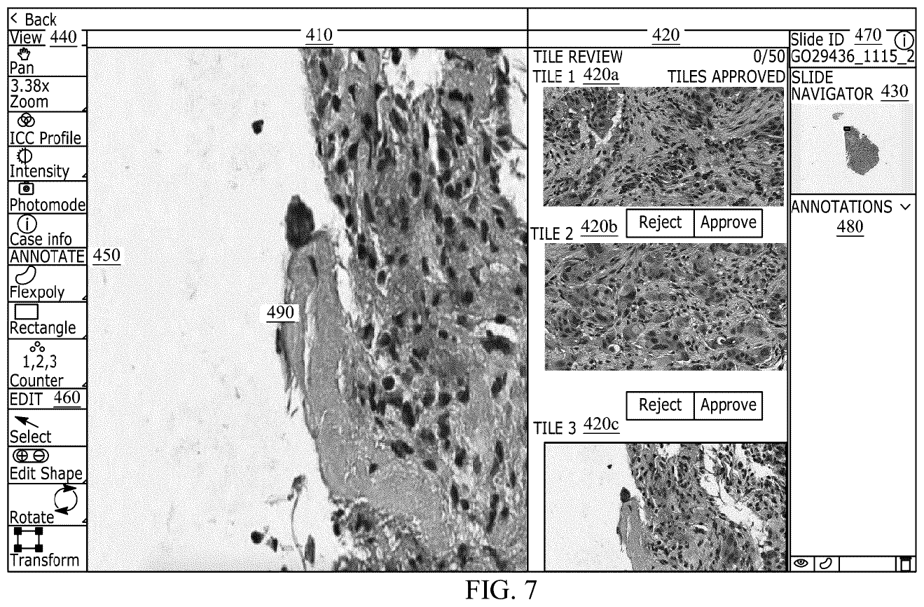

FIG. 7 illustrates another example tile under review by the pathologist. As illustrated in FIG. 7, the pathologist may be reviewing tile 3 420c. The slide navigator 430 may indicate the location of this tile 420c with respect to the whole slide image.

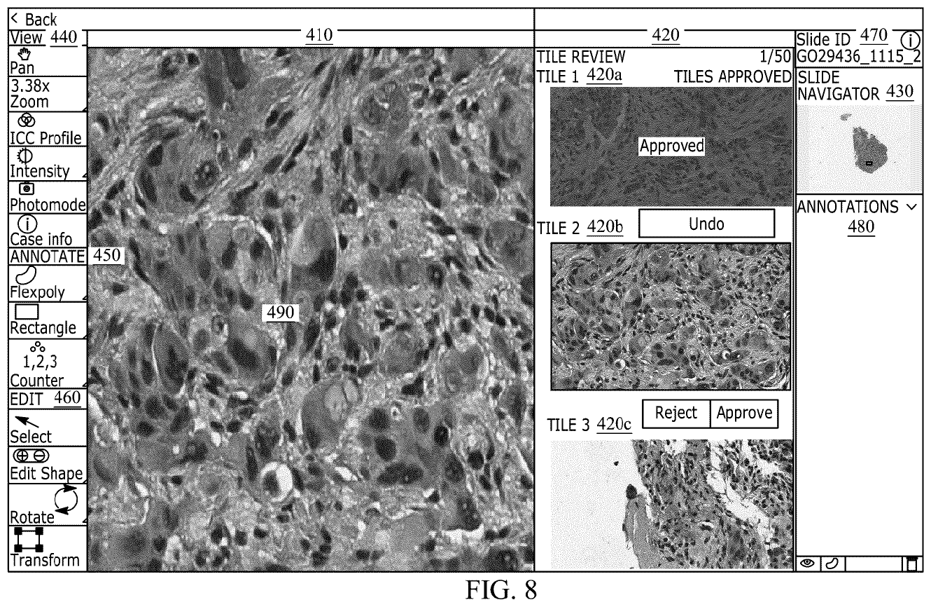

FIG. 8 illustrates an example approval of a tile by the pathologist. As illustrated in FIG. 8, the pathologist may have approved tile 1 420a. As a result, there may be 1 out 50 tiles approved now. The pathologist may undo the approval. The pathologist may be now reviewing tile 2 420b. The slide navigator 430 may indicate the location of tile 2 420b with respect to the whole slide image.

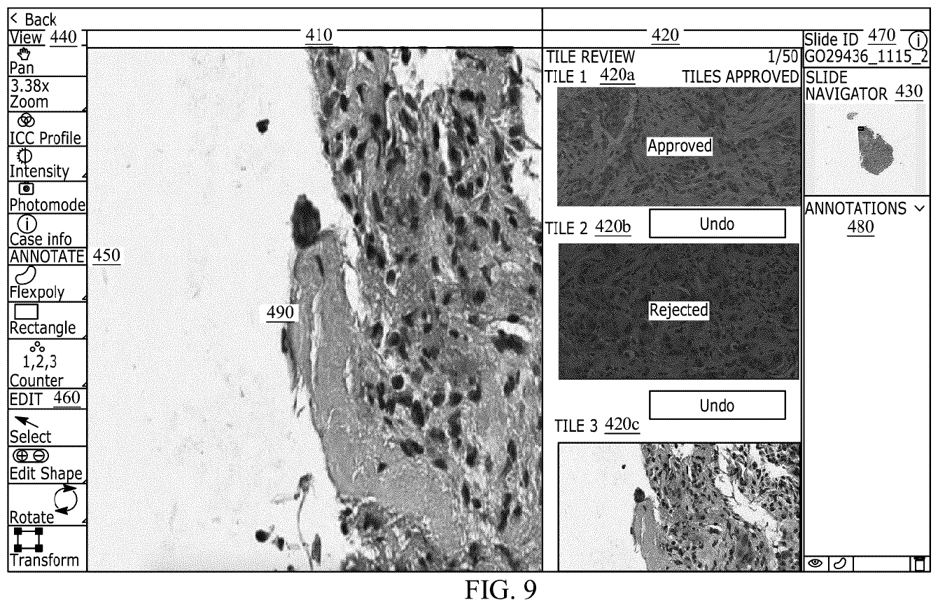

FIG. 9 illustrates an example rejection of a tile by the pathologist. As illustrated in FIG. 9, the pathologist may have rejected tile 2 420b. The pathologist may undo the rejection. The pathologist may be now reviewing tile 3 420c. The slide navigator 430 may indicate the location of tile 3 420c with respect to the whole slide image. Because of the rejection of tile 2 420b, the digital pathology image processing system 110 may replace the rejected tile 2 420b with another tile for the pathologist to review.

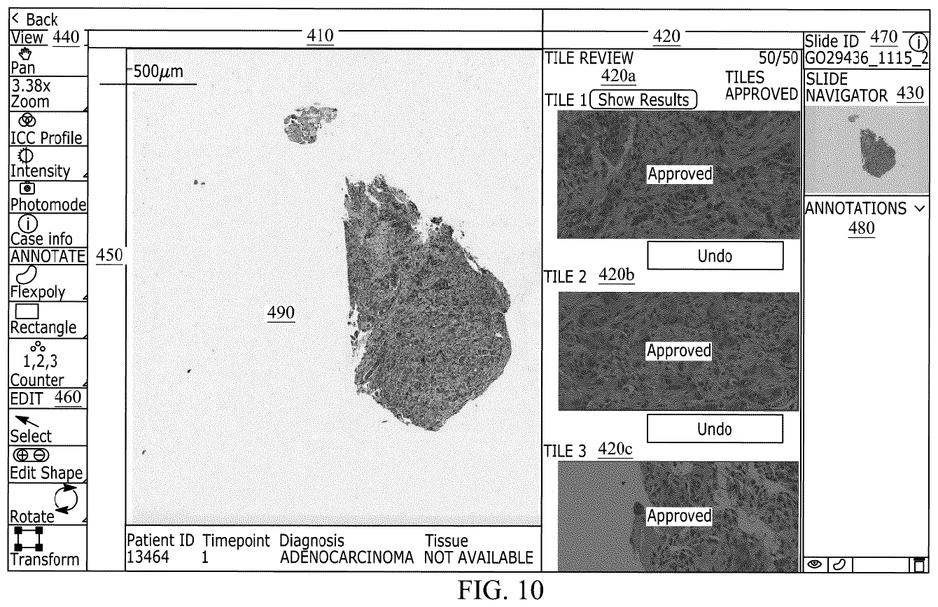

FIG. 10 illustrates an example of minimum number of approved tiles being reached. As illustrated in FIG. 10, there may be now 50 tiles out of the required 50 tiles being approved. The approval of the minimum number of tiles may trigger the statistical analysis by the digital pathology image processing system 110. As a result, the pathologist may select “show results” 1010 to see the analysis results.

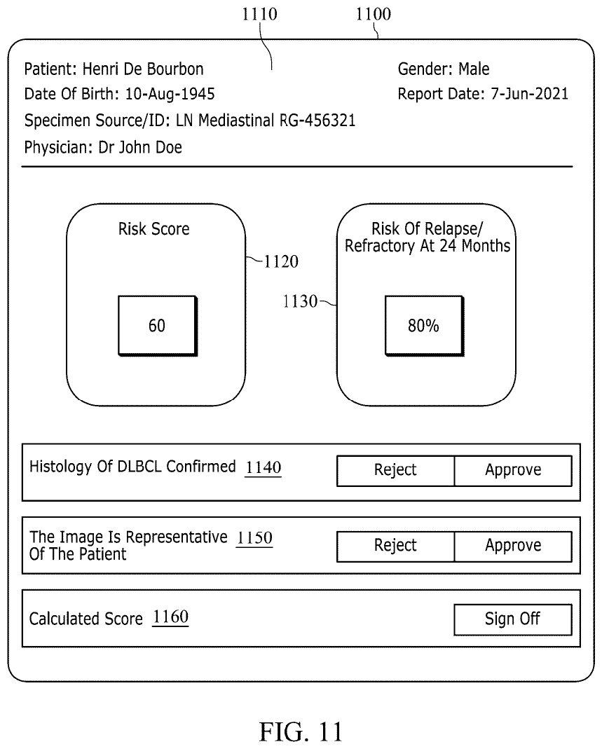

FIG. 11 illustrates an example report 1100 generated by the digital pathology image processing system 110. As an example and not by way of limitation, the report 1100 may comprise patient information 1110 such as patient name, gender, date of birth, report date, specimen source/identifier (ID), and physician. The report may also comprise a risk score 1120 (e.g., 60) and a risk of relapse/refractory at 24 months (e.g., 80%) 1130. The software tool may enable the pathologist to confirm the histology of DLBCL 1140. For example, the pathologist may either reject or approve that the histology is confirmed. The software tool may also enable the pathologist to reject or approve that the image is representative of the patient 1150. The software tool may additionally enable the pathologist to approve the calculated risk score. By approving the calculated risk score, the pathologist may automatically sign off the report.

In particular embodiments, the analysis of slide images based on the aforementioned workflow, i.e., pre-selecting tiles based on machine-learning models and having pathologists review the pre-selected tiles, may also comprise predicting cell of origins of the slide images. In particular embodiments, for predicting cell of origins, the pre-selection of tiles may be by the machine-learning models may be considered as region proposal. The embodiments disclosed here conducted experiments on predicting cell of origins and have the following results. The results show that by integrating region proposal by machine-learning models and pathologist review, the digital pathology image processing system 110 may improve the accuracy of predicting cell of origins over using machine-learning models alone for region proposal. The region proposal result improves after the manual quality-check review where the pathologist rejected some of the regions. The experiments are based on a plurality of (e.g., 97) Goya test set slides that have the manual tumor annotations. The comparison of results measured by AUC (Area under the ROC Curve) is as follows. The AUC for a baseline model that used the manual tumor annotations is 74.3%. The AUC for region proposal without pathologist review is 72.6%. The AUC for region proposal with pathologist review 74.2%. Since the embodiments disclosed herein are not restricted to the manual tumor annotations, we may also report the results on a larger cohort of 129 slides that includes slides without manual tumor annotations. The AUC for region proposal without pathologist review is 75.3%. The AUC for region proposal with pathologist review is 76.7%.

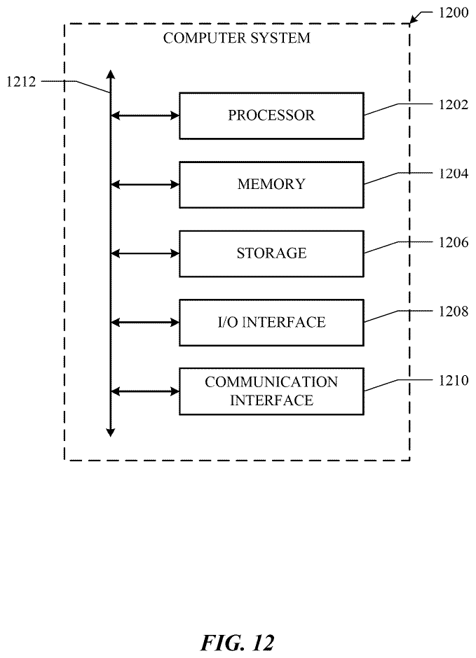

FIG. 12 illustrates an example computer system 1200. In particular embodiments, one or more computer systems 1200 perform one or more steps of one or more methods described or illustrated herein. In particular embodiments, one or more computer systems 1200 provide functionality described or illustrated herein. In particular embodiments, software running on one or more computer systems 1200 performs one or more steps of one or more methods described or illustrated herein or provides functionality described or illustrated herein. Particular embodiments include one or more portions of one or more computer systems 1200. Herein, reference to a computer system may encompass a computing device, and vice versa, where appropriate. Moreover, reference to a computer system may encompass one or more computer systems, where appropriate.

This disclosure contemplates any suitable number of computer systems 1200. This disclosure contemplates computer system 1200 taking any suitable physical form. As example and not by way of limitation, computer system 1200 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1200 may include one or more computer systems 1200; be unitary or distributed; span multiple locations; span multiple machines; span multiple data centers; or reside in a cloud, which may include one or more cloud components in one or more networks. Where appropriate, one or more computer systems 1200 may perform without substantial spatial or temporal limitation one or more steps of one or more methods described or illustrated herein. As an example and not by way of limitation, one or more computer systems 1200 may perform in real time or in batch mode one or more steps of one or more methods described or illustrated herein. One or more computer systems 1200 may perform at different times or at different locations one or more steps of one or more methods described or illustrated herein, where appropriate.

In particular embodiments, computer system 1200 includes a processor 1202, memory 1204, storage 1206, an input/output (I/O) interface 1208, a communication interface 1210, and a bus 1212. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1202 includes hardware for executing instructions, such as those making up a computer program. As an example and not by way of limitation, to execute instructions, processor 1202 may retrieve (or fetch) the instructions from an internal register, an internal cache, memory 1204, or storage 1206; decode and execute them; and then write one or more results to an internal register, an internal cache, memory 1204, or storage 1206. In particular embodiments, processor 1202 may include one or more internal caches for data, instructions, or addresses. This disclosure contemplates processor 1202 including any suitable number of any suitable internal caches, where appropriate. As an example and not by way of limitation, processor 1202 may include one or more instruction caches, one or more data caches, and one or more translation lookaside buffers (TLBs). Instructions in the instruction caches may be copies of instructions in memory 1204 or storage 1206, and the instruction caches may speed up retrieval of those instructions by processor 1202. Data in the data caches may be copies of data in memory 1204 or storage 1206 for instructions executing at processor 1202 to operate on; the results of previous instructions executed at processor 1202 for access by subsequent instructions executing at processor 1202 or for writing to memory 1204 or storage 1206; or other suitable data. The data caches may speed up read or write operations by processor 1202. The TLBs may speed up virtual-address translation for processor 1202. In particular embodiments, processor 1202 may include one or more internal registers for data, instructions, or addresses. This disclosure contemplates processor 1202 including any suitable number of any suitable internal registers, where appropriate. Where appropriate, processor 1202 may include one or more arithmetic logic units (ALUs); be a multi-core processor; or include one or more processors 1202. Although this disclosure describes and illustrates a particular processor, this disclosure contemplates any suitable processor.

In particular embodiments, memory 1204 includes main memory for storing instructions for processor 1202 to execute or data for processor 1202 to operate on. As an example and not by way of limitation, computer system 1200 may load instructions from storage 1206 or another source (such as, for example, another computer system 1200) to memory 1204. Processor 1202 may then load the instructions from memory 1204 to an internal register or internal cache. To execute the instructions, processor 1202 may retrieve the instructions from the internal register or internal cache and decode them. During or after execution of the instructions, processor 1202 may write one or more results (which may be intermediate or final results) to the internal register or internal cache. Processor 1202 may then write one or more of those results to memory 1204. In particular embodiments, processor 1202 executes only instructions in one or more internal registers or internal caches or in memory 1204 (as opposed to storage 1206 or elsewhere) and operates only on data in one or more internal registers or internal caches or in memory 1204 (as opposed to storage 1206 or elsewhere). One or more memory buses (which may each include an address bus and a data bus) may couple processor 1202 to memory 1204. Bus 1212 may include one or more memory buses, as described below. In particular embodiments, one or more memory management units (MMUs) reside between processor 1202 and memory 1204 and facilitate accesses to memory 1204 requested by processor 1202. In particular embodiments, memory 1204 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1204 may include one or more memories 1204, where appropriate. Although this disclosure describes and illustrates particular memory, this disclosure contemplates any suitable memory.

In particular embodiments, storage 1206 includes mass storage for data or instructions. As an example and not by way of limitation, storage 1206 may include a hard disk drive (HDD), a floppy disk drive, flash memory, an optical disc, a magneto-optical disc, magnetic tape, or a Universal Serial Bus (USB) drive or a combination of two or more of these. Storage 1206 may include removable or non-removable (or fixed) media, where appropriate. Storage 1206 may be internal or external to computer system 1200, where appropriate. In particular embodiments, storage 1206 is non-volatile, solid-state memory. In particular embodiments, storage 1206 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1206 taking any suitable physical form. Storage 1206 may include one or more storage control units facilitating communication between processor 1202 and storage 1206, where appropriate. Where appropriate, storage 1206 may include one or more storages 1206. Although this disclosure describes and illustrates particular storage, this disclosure contemplates any suitable storage.

In particular embodiments, I/O interface 1208 includes hardware, software, or both, providing one or more interfaces for communication between computer system 1200 and one or more I/O devices. Computer system 1200 may include one or more of these I/O devices, where appropriate. One or more of these I/O devices may enable communication between a person and computer system 1200. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1208 for them. Where appropriate, I/O interface 1208 may include one or more device or software drivers enabling processor 1202 to drive one or more of these I/O devices. I/O interface 1208 may include one or more I/O interfaces 1208, where appropriate. Although this disclosure describes and illustrates a particular I/O interface, this disclosure contemplates any suitable I/O interface.

In particular embodiments, communication interface 1210 includes hardware, software, or both providing one or more interfaces for communication (such as, for example, packet-based communication) between computer system 1200 and one or more other computer systems 1200 or one or more networks. As an example and not by way of limitation, communication interface 1210 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1210 for it. As an example and not by way of limitation, computer system 1200 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1200 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1200 may include any suitable communication interface 1210 for any of these networks, where appropriate. Communication interface 1210 may include one or more communication interfaces 1210, where appropriate. Although this disclosure describes and illustrates a particular communication interface, this disclosure contemplates any suitable communication interface.

In particular embodiments, bus 1212 includes hardware, software, or both coupling components of computer system 1200 to each other. As an example and not by way of limitation, bus 1212 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1212 may include one or more buses 1212, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

### Example Embodiments

Embodiments disclosed herein may include:
1. A method comprising, by a digital pathology image processing system: accessing a slide image associated with a tissue for a medical analysis; segmenting the slide image into a plurality of tiles; selecting, by one or more machine-learning models based on one or more criteria associated with the medical analysis, one or more tiles from the plurality of tiles; displaying, via a user interface, the one or more selected tiles for user review; receiving, via the user interface, one or more user inputs associated with the one or more tiles; and generating, based on the one or more user inputs and the one or more tiles, an analysis result for the medical analysis.
2. The method of embodiment 1, further comprising: generating a first subset from the plurality of tiles by filtering out one or more first tiles from the plurality of tiles, wherein each of the one or more first tiles comprises an artifact, and wherein the one or more tiles are selected from the first subset.
3. The method of embodiment 2, further comprising: generating a second subset from the first subset by filtering out one or more second tiles from the first subset, wherein each of the one or more second tiles corresponds to an area for the medical analysis, and wherein the one or more tiles are selected from the second subset.
4. The method of embodiment 3, where the medical analysis comprises a tumor analysis, and wherein generating the second subset is based on a tumor detection algorithm, and wherein each tile in the second subset comprises a tumor.
5. The method of any one of embodiments 1-4, wherein the one or more criteria comprise one or more of a high-attention value or a high representativeness of an illness targeted by the medical analysis.
6. The method of any one of embodiments 1-5, wherein the tissue is associated with a patient having a tumor, and wherein the method further comprises: generating, for each of the one or more selected tiles, segmentations comprising nuclei.
7. The method of any one of embodiments 1-6, wherein the medical analysis comprises determining one or more of a recurrence of an illness or a resistance to a treatment.
8. The method of any one of embodiments 1-7, wherein the one or more user inputs comprise one or more of an approval of a tile, a rejection of a tile, or a score for a tile.
9. The method of any one of embodiments 1-8, wherein the one or more user inputs comprise one or more approvals of one or more tiles, wherein the method further comprises: determining a number of the one or more approved tiles reaches a predetermined number, wherein the analysis result is automatically generated based on the number of the one or more approved tiles reaching the predetermined number.
10. The method of any one of embodiments 1-9, wherein the one or more user inputs comprise one or more rejections of one or more tiles, wherein the method further comprises: selecting, by the one or more machine-learning models based on the one or more criteria associated with the medical analysis, one or more additional tiles from the plurality of tiles for the user review.
11. The method of any one of embodiments 1-10, wherein the analysis result comprises one or more of a risk score indicating a likelihood for a recurrence of an illness, a risk score indicating a likelihood for a resistance to a treatment, or a probability indicating a risk of relapse or refractory at a particular time point.
12. The method of any one of embodiments 1-11, further comprising: displaying, via the user interface, one or more locations of the one or more selected tiles with respect to the tissue, respectively.
13. The method of any one of embodiments 1-12, wherein the tissue is stained based on a H & E stain, wherein the method further comprises: generating, by the one or more machine-learning models, a virtual DAB stain for associated with the tissue, wherein the user interface is operable for adjusting the display of each of the one or more selected tiles based on one or more of the H & E stain or the virtual DAB stain.
14. The method of any one of embodiments 1-13, further comprising: receiving, via the user interface, one or more additional user inputs comprising one or more of an approval of the analysis result, an adjustment of the analysis result, or an override of the analysis result; generating, based on the one or more additional user inputs, a medical report; and receiving a sign-off of the medical report.
15. One or more computer-readable non-transitory storage media embodying software that is operable when executed to perform the method of any one of embodiments 1-14.
16. A system comprising: one or more processors; and a non-transitory memory coupled to the processors comprising instructions executable by the processors, the processors operable when executing the instructions to perform the method of any one of embodiments 1-14.

What is claimed is:

1. A method comprising, by a digital pathology image processing system:
accessing a slide image associated with a tissue for a medical analysis;
segmenting the slide image into a plurality of tiles;
selecting, by one or more machine-learning models based on one or more criteria associated with the medical analysis, one or more tiles from the plurality of tiles;
displaying, via a user interface, the one or more selected tiles for user review;
receiving, via the user interface, one or more user inputs associated with the one or more tiles; and
generating, based on the one or more user inputs and the one or more tiles, an analysis result for the medical analysis.

2. The method of claim 1, further comprising:
generating a first subset from the plurality of tiles by filtering out one or more first tiles from the plurality of tiles, wherein each of the one or more first tiles comprises an artifact, and wherein the one or more tiles are selected from the first subset.

3. The method of claim 2, further comprising:
generating a second subset from the first subset by filtering out one or more second tiles from the first subset, wherein each of the one or more second tiles corresponds to an area for the medical analysis, and wherein the one or more tiles are selected from the second subset.

4. The method of claim 3, where the medical analysis comprises a tumor analysis, and wherein generating the second subset is based on a tumor detection algorithm, and wherein each tile in the second subset comprises a tumor.

5. The method of claim 1, wherein the one or more criteria comprise one or more of a high-attention value or a high representativeness of an illness targeted by the medical analysis.

6. The method of claim 1, wherein the tissue is associated with a patient having a tumor, and wherein the method further comprises:
generating, for each of the one or more selected tiles, segmentations comprising nuclei.

7. The method of claim 1, wherein the medical analysis comprises determining one or more of a recurrence of an illness or a resistance to a treatment.

8. The method of claim 1, wherein the one or more user inputs comprise one or more of an approval of a tile, a rejection of a tile, or a score for a tile.

9. The method of claim 1, wherein the one or more user inputs comprise one or more approvals of one or more tiles, wherein the method further comprises:
determining a number of the one or more approved tiles reaches a predetermined number, wherein the analysis result is automatically generated based on the number of the one or more approved tiles reaching the predetermined number.

10. The method of claim 1, wherein the one or more user inputs comprise one or more rejections of one or more tiles, wherein the method further comprises:
selecting, by the one or more machine-learning models based on the one or more criteria associated with the medical analysis, one or more additional tiles from the plurality of tiles for the user review.

11. The method of claim 1, wherein the analysis result comprises one or more of a risk score indicating a likelihood for a recurrence of an illness, a risk score indicating a likelihood for a resistance to a treatment, or a probability indicating a risk of relapse or refractory at a particular time point.

12. The method of claim 1, further comprising:
displaying, via the user interface, one or more locations of the one or more selected tiles with respect to the tissue, respectively.

13. The method of claim 1, wherein the tissue is stained based on a H & E stain, wherein the method further comprises:
generating, by the one or more machine-learning models, a virtual DAB stain for associated with the tissue, wherein the user interface is operable for adjusting the display of each of the one or more selected tiles based on one or more of the H & E stain or the virtual DAB stain.

14. The method of claim 1, further comprising:
receiving, via the user interface, one or more additional user inputs comprising one or more of an approval of the analysis result, an adjustment of the analysis result, or an override of the analysis result;
generating, based on the one or more additional user inputs, a medical report; and
receiving a sign-off of the medical report.

15. One or more computer-readable non-transitory storage media embodying software that is operable when executed to:
access a slide image associated with a tissue for a medical analysis;
segment the slide image into a plurality of tiles;
select, by one or more machine-learning models based on one or more criteria associated with the medical analysis, one or more tiles from the plurality of tiles;
display, via a user interface, the one or more selected tiles for user review;
receive, via the user interface, one or more user inputs associated with the one or more tiles; and
generate, based on the one or more user inputs and the one or more tiles, an analysis result for the medical analysis.

16. The media of claim 15, wherein the software is further operable when executed to:
generate a first subset from the plurality of tiles by filtering out one or more first tiles from the plurality of tiles, wherein each of the one or more first tile comprises an artifact, and wherein the selected one or more tiles are selected from the first subset.

17. The media of claim 16, wherein the software is further operable when executed to:
generate a second subset from the first subset by filtering out one or more second tiles from the first subset, wherein each of the one or more second tile corresponds to an area for the medical analysis, and wherein the selected one or more tiles are selected from the second subset.

18. The media of claim 15, wherein the one or more criteria comprise one or more of a high-attention value or a high representativeness of an illness targeted by the medical analysis.

19. A system comprising: one or more processors; and a non-transitory memory coupled to the processors comprising instructions executable by the processors, the processors operable when executing the instructions to:
access a slide image associated with a tissue for a medical analysis;
segment the slide image into a plurality of tiles;
select, by one or more machine-learning models based on one or more criteria associated with the medical analysis, one or more tiles from the plurality of tiles;
display, via a user interface, the one or more selected tiles for user review;
receive, via the user interface, one or more user inputs associated with the one or more tiles; and
generate, based on the one or more user inputs and the one or more tiles, an analysis result for the medical analysis.

20. The system of claim 19, wherein the one or more criteria comprise one or more of a high-attention value or a high representativeness of an illness targeted by the medical analysis.
